(19) United States
(12) Patent Application Publication
(10) Pub. No.: US 2015/0151011 A1
(43) Pub. Date: Jun. 4, 2015

(54) Preparation of Technetium-99M Tricarbonyl Labeled Glycine Monomer or Oligomer Containing Probes That Have Biomolecules and Its Application as Imaging Complex-Composition

(71) Applicant: KOREA ATOMIC ENERGY RESEARCH INSTITUTE, Daejeon (KR)

(72) Inventors: Beom Su Jang, Daejeon (KR); Sang Hyun Park, Jeollabuk-do (KR); Joo-Sang Lee, Daegu (KR); Jong-Kook Rho, Busan (KR)

(73) Assignee: KOREA ATOMIC ENERGY RESEARCH INSTITUTE, Daejeon (KR)

(21) Appl. No.: 14/399,431

(22) PCT Filed: May 13, 2013
(86) PCT No.: PCT/KR2013/004232

§ 371 Date: Nov. 6, 2014

(30) Foreign Application Priority Data
May 14, 2012 (KR) 10-2012-0051156

(51) Int. Cl. A61K 51/08; C07K 7/06; C07K 1/13; C07K 5/083; C07B 59/00; C07C 227/16

(52) U.S. Cl. CPC A61K 51/082; C07B 59/001; C07C 227/16; C07K 1/13; C07K 5/0806; C07K 7/06; A61K 51/085; C07B 2200/05

(57) ABSTRACT

Disclosed is a technetium-99m-labeled glycine oligomer associated with imaging probes for biomolecules of interest. The glycine oligomer can be readily synthesized in a single process using an automated peptide synthesizer. The technetium-99m tricarbonyl-labeled glycine oligomers can be useful as a radiotracer for gamma or SPECT imaging apparatus. The technetium-99m tricarbonyl-labeled glycine oligomers can be applied to various peptidyl biomolecules such as RGD peptide, somatostatin, neurotensin, etc., and exhibit rapid renal clearance without being excessively retained within the body.

### TECHNICAL FIELD

The present invention relates to a method for preparing a technetium-99m tricarbonyl-labeled glycine monomer or oligomer in a single process using automated peptide synthesis, and an imaging contrast composition containing the technetium-99m tricarbonyl-labeled glycine monomer or oligomer.

### BACKGROUND ART

In an age where perfect tumor regulation is one of the main objectives of modern medicine, a scintigraphic detection and therapeutic technique which can non-invasively visualize the state of a tumor at a molecular level using a radioactive tracer reactive specifically to a target molecule occupies a very important position, as it allows for the early diagnosis and therapy of tumor. For example, [¹⁸F]-FDG has been widely used in tumor diagnosis as a radiopharmaceutical for positron emission tomography (PET) and has provided information on the angiogenesis and metastasis of tumor. However, it suffers from the disadvantages of being unable to diagnose cerebral tumors and to discriminate between a tumor and inflammation.

Integrins are heterodimeric transmembrane glycoproteins that consist of 19 α-subunits and 8 β-subunits and play a key role in tumor-induced angiogenesis, tumor invasions and metastases. Among them, αvβ3, also known as a vitronectin receptor, have been the target of potential radiotracer for providing information about the tumor progression and the malignancy. This integrin is highly expressed on proliferating endothelial cells and solid tumor cells, whereas it is not expressed on quiescent endothelial cells. Arg-Gly-Asp (RGD) peptide is a key binding moiety that has been shown to bind specifically to integrin receptors. This tripeptidyl RGD (arginylglycylaspactic acid) sequence is involved in various intercellular interactions at the cell membrane, and thus is widely studied for the diagnosis and therapy of various tumors and diseases. The cyclic peptide structure, cRGD (cyclic Arg-Gly-Asp) with higher affinity, was developed as an antagonist to the αvβ3 integrin, based on the fact that the RGD, and cRGD peptide derivatives labeled with various radioactive isotopes have been used as integrin-targeted radiotracers for use in molecular imaging studies on cancer-induced angiogenesis. For a potential use of cRGD derivatives as diagnostic radiopharmaceuticals, various gamma-emitting isotopes including technetium-99m, iodide-123, indium-111, gallium-67/68 and copper-64 have been used for SPECT or PET imaging. Monosaccharides may be attached to a radiotracer to induce rapid release from the liver, thereby improving the pharmacokinetic behavior of the RGD-based radiotracer. In addition, two or four cRGD molecules were attached to one radiotracer to increase affinity for the integrin (refer to: Haubner, R., Wester, H-J., Weber, W. A., Mang, C., Ziegler, S. I., Goodman, S. L., Senekowitsch-Schmidtke, R., Kessler, H., Schwaiger, M., Cancer Res., 2001, 61:1781-1785; Jeong, J. M., Hong, M. K., Chang, Y. S., Lee, Y. S., Kim, Y. J., Cheon, G. J., Lee, D. S., Chung, J. K., Lee, M. C., J. Nucl. Med., 2008, 49:830-836; Chen, W., Park, R., Tohme, M., Shahinian, A. H., Bading, J. R., Conti, P. S. Bioconjugate Chem., 2004, 15:41-49; Liu, S., Hsieh, W. Y., Kim, Y. S., Mohammel, S. I., Bioconjugate Chem., 2005, 16:1580-1588; Haubner, R., Wester, H-J., Reuning, U., Senekowitsch-Schmidtke, R., Diefenbach, B., Kessler, H., Stocklin G., Schwaiger, M., J. Nucl. Med., 1999, 40:1061-1071; Janssen, M., Oyen, W. J. G., Dijkgraaf, I., Massuger, L. F. A. G., Frielink, C., Edwards, D. S., Rajopadyhe, M., Boonstra, H., Corsten, F. H. M., Boerman, O. C., Cancer Res., 2002, 62:6146-6151; Wu, Y., Zhang, X., Xiong, Z., Cheng, Z., Fisher, D. R., Liu, S., Gambhir, S. S., Chen, X., J. Nucl. Med., 2005, 46:1707-1718.).

Since biologically active molecules mediate physiological or pathological actions in vivo, evaluation for their in vivo behaviors have important significance in understanding physiological and pathological phenomena and diagnosing and treating relevant diseases. With the advance of molecular imaging techniques, in vivo behaviors of various biologically active molecules can be visualized using highly sensitive radioisotopes and such molecules can then be clinically applied. For example, [¹⁸F]-FDG(2-fluoro-2-deoxy-D-glucose) is widely applied to the quantification of in vivo metabolic rates of glucose and to tumor diagnosis. Since the positron-emitting radioisotopes, such as F-18 and C-11, have short half-lives (110 and 20 min, respectively), they are suitable for longitudinal PET imaging at early time points. While F-18 and C-11 are particularly useful for PET, technetium-99m is the most widely used radionuclide for single photon emission computed tomography (SPECT) due to its optimal nuclear properties (half life=6 h, 140 keV gamma photons) and its convenient availability from commercial generator at low costs. This makes it attractive to use of technetium-99m for developing a radiotracer for tumor imaging.

In conventional techniques utilizing gamma rays emitted from technetium-99m, peptide sequences of biologically active compounds are usually conjugated with chelators. After peptides are synthesized, a chelator is chemically conjugated into the peptides at a terminal amino acid residue or a specific amino acid sequence. In addition to these two steps, an additional step such as purification may be required, which results in a decrease in synthesis yield.

When radioactive ligands for imaging are not excreted from the body, non-specific signals are generated within the body, which result in decreasing a signal-to-noise ratio and thus detection sensitivity. Furthermore, the retained radioactive ligands within the body increase the risk of excessively exposing the body to the radiation. Thus, efforts have been made to overcome these problems.

### DISCLOSURE

### Technical Problem

Leading to the present invention, the present inventors, aiming to overcome the problems encountered in the prior art, conducted intensive and thorough research into an imaging contrast agent that can be readily conjugated into the technetium-99m tricarbonyl precursor and exhibit renal clearance, which resulted in the finding that glycine, whether in the form of a monomer or an oligomer, can be readily combined with the technetium-99m tricarbonyl precursor, and that the resulting technetium-99m tricarbonyl glycine complex acts as a bifunctional chelator and is rapidly cleared from the blood.

It is therefore an object of the present invention to provide a method for preparing a technetium-99m tricarbonyl-labeled glycine monomer or oligomer, and an imaging contrast composition comprising the technetium-99m tricarbonyl-labeled glycine monomer or oligomer.

However, the aims to be achieved in the present invention are not limited to the above-mentioned objects, and the other objects, features and advantages of the present invention will be more clearly understood from the following detailed description.

### Technical Solution

In order to accomplish the above object, the present invention provides an imaging contrast composition containing a technetium-99m-labeled glycine monomer or oligomer.

In addition, the present invention provides a method for preparing a technetium-99m-labeled glycine monomer or oligomer technetium-99m, comprising: synthesizing a technetium-99m tricarbonyl precursor, and labeling a glycine monomer or oligomer with the technetium-99m tricarbonyl precursor.

### Advantageous Effects

The technetium-99m tricarbonyl-labeled glycine oligomer can be applied to various peptidyl biomolecules such as RGD peptide, somatostatin, neurotensin, etc., and exhibit rapid renal clearance without being excessively retained within the body.

### DESCRIPTION OF DRAWINGS

FIG. 1 shows HPLC profiles of technetium-99m (A), a technetium-99m tricarbonyl precursor (B), a technetium-99m tricarbonyl glycine (C), a technetium-99m tricarbonyl glycine trimer (D), and a technetium-99m tricarbonyl glycine pentamer (E);

FIG. 2 shows SPECT images of ICR mice in which a technetium-99m tricarbonyl precursor (A), technetium-99m tricarbonyl glycine (B), a technetium-99m tricarbonyl glycine trimer (C), a technetium-99m tricarbonyl glycine pentamer (D) are distributed.

FIG. 3 shows HPLC profiles of a technetium-99m tricarbonyl precursor (A), technetium-99m tricarbonyl AGRGDS (B: RGD peptide), technetium-99m tricarbonyl GGGAGRGDS (C: RGD peptide containing a glycine trimer), technetium-99m tricarbonyl RRPIL (D: Neurotensin (8-13) peptide), and technetium-99m tricarbonyl Neurotensin (8-13) (E: RGD peptide containing a glycine trimer).

### BEST MODE

The present invention pertains to the use of glycine monomer or oligomer that can be synthesized in a single process using an automated peptide synthesizer and that can be effectively applied to a radiotracer in view of cost and time. More particularly, the present invention addresses an image contrast composition for imaging contrast containing a technetium-99m-labeled glycine monomer or oligomer.

In addition, the present invention addresses a method for preparing a technetium-99m-labeled glycine monomer or oligomer, comprising:
a) synthesizing a technetium-99m tricarbonyl precursor; and
b) labeling a glycine monomer or oligomer with the technetium-99m tricarbonyl precursor.

As will be explained in detail in the following Example section, the present inventors labeled a glycine monomer, trimer (Gly(3)) and/or pentamer (Gly(5)) with a technetium-99m tricarbonyl precursor to afford technetium-labeled tricarbonyl glycines (Example 1) which were identified to be hydrophilic as determined by a octanol-water partition coefficient method (Example 3), and to be released rapidly from the body through the kidney as determined by tomography (Example 4).

According to the present invention, glycine may be added to known targeted peptides by automated peptide synthesis. In greater detail, the synthesis of peptides may be carried out using the following method in an embodiment of the present invention, but is not limited thereto. Peptides were synthesized by Fmoc solid phase peptide synthesis (SPPS) using ASP48S (Peptron Inc.), and purified by the reverse phase HPLC using a Vydac Everest C18 column (250 mm×22 mm, 10 μm). Elution was carried out with a water-acetonitrile linear gradient (10 to 75% (v/v) of acetonitrile) containing 0.1% (v/v) trifluoroacetic acid. A molecular weight of the purified peptide was confirmed using an LC/MS (Agilent HP1100 series).

Fmoc SPPS is designed to couple amino acid units one by one from the C-terminus. NH₂—Ser(tBu)-2-chloro-Trityl Resin, or NH₂-Leu-2-chloro-Trityl Resin, was used, in which the first amino acid of the C-terminal of the peptide was attached to a resin. All the amino acids used in the peptide synthesis were N-protected by Fmoc (Fluorenylmethyloxycarbonyl) while residues were protected by Trt, Boc, t-Bu, Pbf, and the like, which were all removed in acid. For example, Fmoc-Ala-OH, Fmoc-Asp(OtBu)-OH, Fmoc-Arg(Pbf)-OH, Fmoc-Gln(Trt)-OH, Fmoc-Pro-OH, Fmoc-Met-OH, Fmoc-His(Trt)-OH, Fmoc-Ile-OH, Fmoc-Gly-OH, Fmoc-Lys(Boc)-OH, Fmoc-Ser(tBu)-OH, Fmoc-Thr(tBu)-OH, and Fmoc-Tyr(tBu)-OH may be employed. As a coupling agent, HBTU (2-(1H-Benzotriazole-1-yl)-1,1,3,3-tetamethylaminium hexafluorophosphate)/HOBt (N-Hydroxxybenzotriazole)/NMM (4-Methylmorpholine) was used. The removal of the Fmoc group is achieved using 20% piperidine in DMF. The final cleavage of the protein from the resin and the removal of permanent protecting groups was performed with a mixture of 95:3:2 TFA (trifluoroacetic acid): TIS (triisopropylsilane): H₂O. An RGD or a Neurotensin (8-13) peptide sequence which had been added with a glycine trimer in this manner was observed to be conjugated with a technetium-labeled precursor at higher efficiency, compared to the RGD or the Neurotensin (8-13) peptide itself (Example 2). Thus, the technetium-99m tricarbonyl-labeled glycine monomer or oligomer of the present invention can be further conjugated to a targeted peptide.

As used herein, the term “targeted peptide” refers to a biologically active peptide that is apt to bind to a specific site of cells. Examples of the targeted peptide include, but are not limited to, the RGD peptide (arginylglycylaspartic acid), neurotensin, somatostatin, angiotensin, luteinizing hormone releasing hormone (LHRH), insulin, oxytocin, Neurokinin-1 (NK-1), vasoactive intestinal peptide (VIP), substance P (SP), neuropeptide Y (NPY), endothelin A, endothelin B, bradykinin, interleukin-1, epidermal growth factor (EGF), cholecystokinin (CCK), galanin, melanocyte-stimulating hormones (MSH), lanreotide, octreotide, and arginine-vasopressin, with preference for RGD (arginylglycylaspartic acid) or neurotensin.

The imaging contrast composition according to the present invention may be administered parenterally, for example, by bolus injection, intravenous injection or intraarterial injection. In order to image the lung, the contrast composition may be administered using a spray or an aerosol. An oral or rectal route may be taken to administer the contrast composition. However, the present invention is not limited to these routes. So long as it is well known in the art, any administration method may be taken in the present invention. In this regard, parenteral dosage forms of the imaging contrast composition must be free of germs, biologically unacceptable substances, and paramagnetic, superparamagnetic, ferromagnetic, or ferromagnetic impurities. The composition of the present invention may be used in combination with a preservative, an antibacterial agent, a buffer and an antioxidant typically used in a non-oral solution, an excipient, and an MR contrast agent. Also, the composition may comprise another additive if it does not interfere with the production, storage or use of the final product. The effective amount of the imaging contrast composition of the present invention is dependent on various factors including the patient's state and age, the severity of disease, formulation forms of the contrast agent, the route of administration, and time of administration, which is already apparent to those skilled in the art. Typically, the amount of a contrast agent administered at a dose of from 1 to 1.000 mg/kg of weight and preferably at a dose of from 3 to 300 mg/kg of weight.

### MODE FOR INVENTION

A better understanding of the present invention may be obtained through the following examples which are set forth to illustrate, but are not to be construed as limiting, the present invention.

### Preparation Example 1

### Preparation of Technetium-99m-Labeled Glycines

Technetium-99m-labeled glycine monomer or oligomers according to the present invention were prepared from the following materials.

\textcircled{1} Materials: Carbon monoxide (99.5%) was provided from Daehan Gas (Seoul, Korea) and purified using an oxygen trap before use. Technetium-99m was produced as pertechnetate using Unitech Tc-99m generator (Samyoung Unitech. Co. Ltd., Korea) in 0.9% sodium chloride. glycin, Gly-Gly-Gly (glycine trimer), and Gly-Gly-Gly-Gly-Gly (glycine pentamer) were purchased from Sigma Chemical Co. (St. Louis, USA).
\textcircled{2} Animal test: Female germ-free ICR mice (7 weeks old) were purchased from Orient, Inc. (Seoul, Korea) and acclimated for one week before use in experiments. The mice were maintained at a relative humidity of 50±5% at a temperature of 23±2° C. on a 12-h light-dark cycle, allowed to have access to food and water ad libitum, and acclimated for at least 1 week prior to usage. All animal experiments were conducted after approval by the Institute Animal Care and Use Committee of the Korean Atomic Energy Research Institute.

### Example 1

### Preparation of Technetium-99m Tricarbonyl Glycine Complex

A technetium-99m tricarbonyl precursor was applied to glycine monomer and oligomers to afford technetium-99m tricarbonyl glycines.

### <1-1> Synthesis of Technetium-99m Tricarbonyl Precursor

[^{99m}Tc(CO)₃(H₂O)₃]⁺ was prepared by adding 1 ml of ^{99m}TcO₄⁻ (10 mCi) to a 5 ml of bottle containing potassium boranocarbonates (5.9 mg), sodium tetraborate decahydrates (2.85 mg), sodium tartrate dehydrate (8.5 mg), and sodium carbonate (7.15 mg) in a commercially available generator, and the solution was heated for 30 min in boiling water under a nitrogen gas condition. The technetium-99m tricarbonyl precursor was examined for labeling yield and stability using reversed-phase high performance liquid chromatography.

For HPLC, the Agilent 1200 series (Agilent Technologies, Waldbronn, Germany), equipped with a vacuum degasser, a binary pump, a temperature-controlling autosampler, a column oven, a UV-Vis detector and a radioactive dating meter was employed. HPLC was performed at ambient temperature on a Nucleosil C18 column (5 micron, 3.0×250 mm, Supelco Inc., PA, USA), eluting with methanol (solvent B) and 0.05 M TEAP (solvent A). The mobile phase was adjusted into a pH of 2.3 with trichloroacetic acid. In HPLC, the elution was carried out with 100% A at the time from 0 to 5 min; a linear gradient of from 75% A/25% B to 100% A/0% B at the time from 5 to 8 min, a linear gradient of from 66% A/34% B to 75% A/25% B at the time from 8 to 11, a linear gradient of from 0% A/100% B to 66% A/34% B at the time from 11 to 22 min, and 100% B at the time from 22 to 25 min. The elution solvent was loaded at a flow rate of 0.6 ml/min in an aliquot of 10 μl.

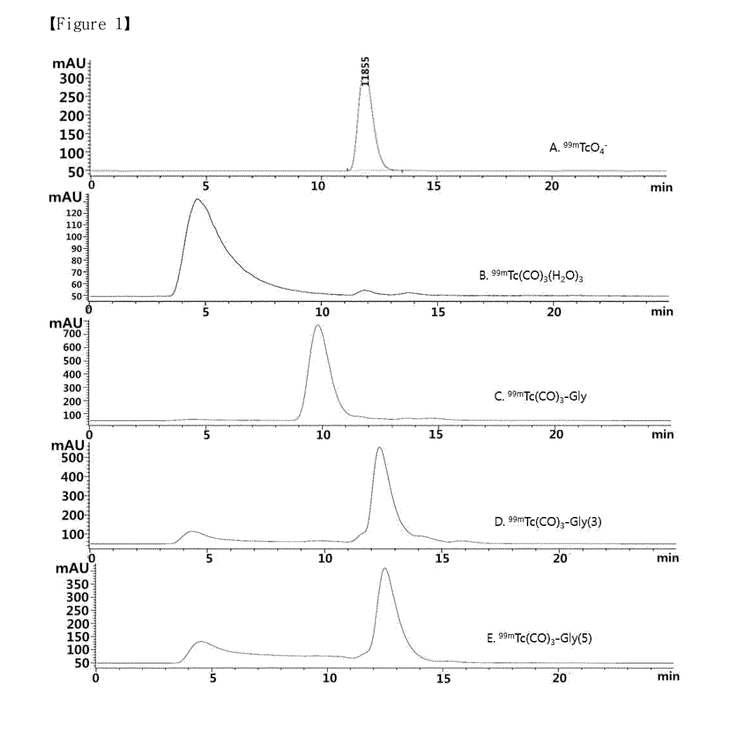

As can be seen in FIG. 1, [^{99m}Tc(CO)₃(H₂O)₃]⁺ was produced at such a high yield (>95%) as to require no additional purification steps.

<1-2> Labeling with Technetium-99m Tricarbonyl Precursor

The technetium-99m tricarbonyl precursor was applied to a glycine monomer or glycine oligomers. In this regard, 1 ml of the technetium-99m tricarbonyl precursor synthesized in Example 1-1 was added to 50 μl of a glycine monomer, a glycine trimer or a glycine pentamer (10 mg/ml in saline) in a reactor and then heated at 75° C. for 30 min. Labeling yield was determined using radio-HPLC.

The measurements are given in FIG. 1. As seen in the chromatograms of FIG. 1, all of the glycine monomer, the glycine trimer, and the glycine pentamer were labeled with the technetium-99m tricarbonyl at a yield of 80% or higher. Upon HPLC, a single peak was detected at 10.3 min for technetium-99m tricarbonyl-glycin while the technetium-99m tricarbonyl core was eluted at 4 min. Thus, glycine and glycine oligomers were readily labeled with the technetium-99m tricarbonyl precursor at high yield.

### Example 2

### Effect of Glycine Trimer Technetium-99m Tricarbonyl-Labeled RGD and Neurotensin (8-13)

A glycine trimer was applied as a terminal sequence to a targeted peptide, and examined for effect on labeling with the technetium-99m tricarbonyl precursor. For this, prototype peptides (AGRGDS and RRPYIL), and glycine trimer-added peptides (GGGAGRGDS and GGGRRPYIL) were synthesized using the following automated peptide synthesis method.

### <2-1> Synthesis of AGRGDS

AGRGDS was synthesized in the following automated peptide synthesis manner.

1) Coupling step: A protected amino acid (8 eqs.) and a coupling reagent HBTU (8 eqs.)/HOBt (8 eqs.)/NMM (16 eqs.) in DMF was added to and reacted with NH₂—Ser(tBu)-2-chloro-Trityl Resin at room temperature for 2 hrs, followed by sequentially washing with DMF, MeOH, and DMF in that order.
2) Fmoc deprotection step: After the coupling step, 20% piperidine in DMF was added to and reacted with the resin at room temperature for 5 min. The procedure was repeated twice before sequentially washing with DMF, MeOH, and DMF.
3) The steps 1) and 2) were repetitively conducted to finally synthesize the peptide scaffold (NH₂-A-G-R(Pbf)-G-D(tBu)-S(tBu)-2-chloro-Trityl Resin), followed by separating the peptide from the resin using a mixture of 95:3:2 TFA:TIS:H₂O.
4) The reaction mixture was added with cooling diethyl ether, and centrifuged to precipitate the peptide. After purification through HPLC, the peptide was examined for mass using MS, and freeze dried.

### <2-2> Synthesis of GGGAGRGDS

GGGAGRGDS was synthesized using the following automated peptide synthesis method.

1) Coupling step: A protected amino acid (8 eqs.) and a coupling reagent HBTU (8 eqs.)/HOBt (8 eqs)/NMM (16 eqs.) in DMF were added and reacted with NH₂-Ser(tBu)-2-chloro-Trityl Resin at room temperature for 2 hrs, followed by sequentially washing with DMF, MeOH, and DMF in that order.
2) Fmoc deprotection step: After the coupling step, 20% piperidine in DMF was added to and reacted with the resin at room temperature for 5 min. The procedure was repeated twice before sequentially washing with DMF, MeOH, and DMF.
3) The steps 1) and 2) were repetitively conducted to finally synthesize the peptide scaffold (NH₂-G-G-G-A-G-R(Pbf)-G-D(tBu)-S(tBu)-2-chloro-TritylResin), followed by separating the peptide from the resin using a mixture of 95:3:2 TFA:TIS:H₂O.
4) The reaction mixture was added with cooling diethyl ether, and centrifuged to precipitate the peptide. After purification through HPLC, the peptide was examined for mass using MS, and freeze dried.

### <2-3> Synthesis of RRPYIL

RRPYIL was synthesized using the following automated peptide synthesis method.

1) Coupling step: A protected amino acid (8 eqs.) and a coupling reagent HBTU (8 eqs.)/HOBt (8 eqs)/NMM (16 eqs.) in DMF were added and reacted with NH₂-Leu-2-chloro-Trityl Resin at room temperature for 2 hrs, followed by sequentially washing with DMF, MeOH, and DMF in that order.
2) Fmoc deprotection step: After the coupling step, 20% piperidine in DMF was added to and reacted with the resin at room temperature for 5 min. The procedure was repeated twice before sequentially washing with DMF, MeOH, and DMF.
3) The steps 1) and 2) were repetitively conducted to finally synthesize the peptide scaffold (NH₂—R(Pbf)-R(Pbf)-P—Y(tBu)-I-L-2-chloro-TritylResin), followed by separating the peptide from the resin using a mixture of 95:3:2 TFA:TIS:H₂O.
4) The reaction mixture was added with cooling diethyl ether, and centrifuged to precipitate the peptide. After purification through HPLC, the peptide was examined for mass using MS, and freeze dried.

### <2-4> Synthesis of GGGRRPYIL

GGGRRPYIL was synthesized using the following automated peptide synthesis method.

1) Coupling step: A protected amino acid (8 eqs.) and a coupling reagent HBTU (8 eqs.)/HOBt (8 eqs)/NMM (16 eqs.) in DMF were added and reacted with NH₂-Leu-2-chloro-Trityl Resin at room temperature for 2 hrs, followed by sequentially washing with DMF, MeOH, and DMF in that order.
2) Fmoc deprotection step: After the coupling step, 20% piperidine in DMF was added to and reacted with the resin at room temperature for 5 min. The procedure was repeated twice before sequentially washing with DMF, MeOH, and DMF.
3) The steps 1) and 2) were repetitively conducted to finally synthesize the peptide scaffold (NH₂-G-G-G-R(Pbf)-R(Pbf)-P—Y(tBu)-I-L-2-chloro-TritylResin), followed by separating the peptide from the resin using a mixture of 95:3:2 TFA:TIS:H₂O.
4) The reaction mixture was added with cooling diethyl ether, and centrifuged to precipitate the peptide. After purification through HPLC, the peptide was examined for mass using MS, and freeze dried.
<2-5> Labeling of the Synthetic Peptides with Technetium-99m Tricarbonyl and Analysis Thereof

The peptides synthesized in Examples 2-1 to 2-4 were labeled with technetium-99m tricarbonyl and analyzed in the same manner as in Example 1.

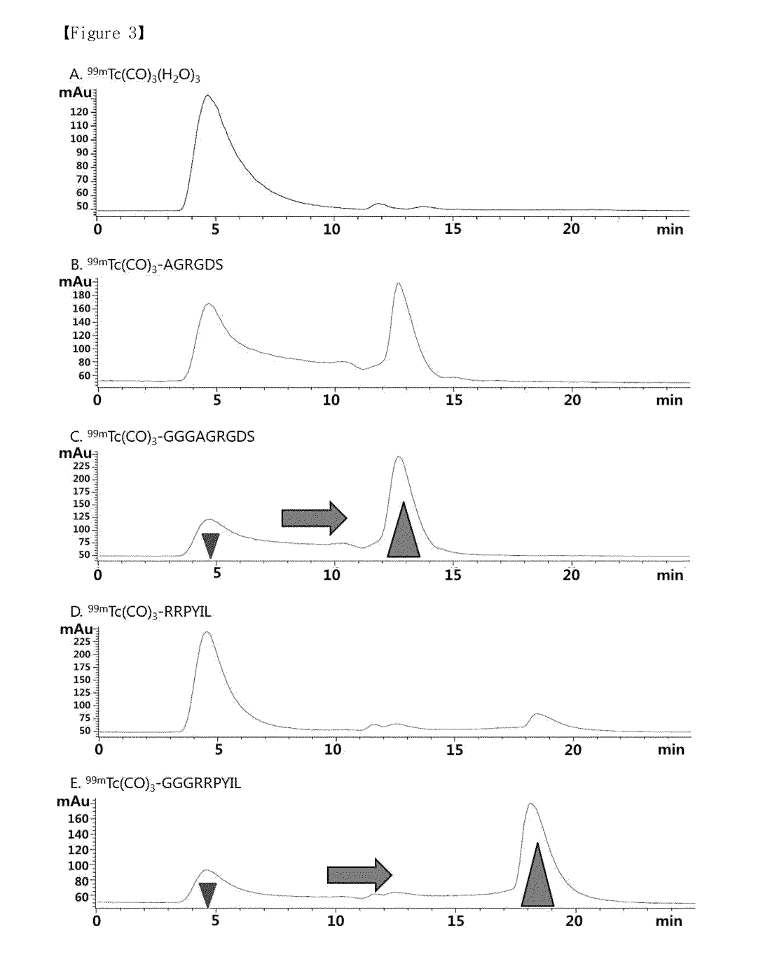

The results are given in FIG. 3. As can be seen in FIG. 3, the glycine trimer-added peptides GGGAGRGDS and GGGRRPYIL were observed to be labeled with technetium-99m tricarbony at higher yield, compared to the prototypes AGRGDS and RRPYIL themselves.

These data indicate that various biologically active compounds, when added with a glycine trimer, can be readily labeled with technetium-99m tricarbonyl.

### Example 3

### Octanol-Water Partition Coefficient of Technetium-99m Tricarbonyl Glycines

In order to predict the in vivo residence and degradation rate of technetium-99m tricarbonyl glycin complexes, octanol-water partition coefficients were measured in triplicate as follows. To a mixture of 500 μl of nitrogen-purged 0.05 M PBS (phosphate-buffered saline, pH 7.4) and 500 μl of n-octanol was added 10 μl of technetium-99m tricarbonyl glycine, glycine trimer, or glycine pentamer, followed by vortexing for 3 min. Centrifugation at 3,000×g for 5 min in a SORVALL FRESCO centrifuge (Asheville, N.C., USA) gave two distinct phases. From each of the PBS layer and the octanol layer was taken 20 μl which was then measured for radioactivity using a well-type NaI (TI) scintillation detector. The coefficient is calculated according to the following equation.

Log p=log(octanol(cpm)/water(cpm)) <Equation>

Octanol-water partition coefficients of the technetium-99m tricarbonyl-glycine monomer and oligomers are given in Table 1, below. They were observed to have an n-octanol/buffer partition coefficient of from about −0.5 to −16.

TABLE 1

| | Octanol/Water Partition |
|---|---|
| Compound | Coeffi. (Kow logP) |
| technetium-99m tricarbonyl-glycine | −0.48 ± 0.00 |
| technetium-99m tricarbonyl-glycine | −1.53 ± 0.02 |
| trimer | |
| technetium-99m tricarbonyl-glycine | −1.50 ± 0.01 |
| pentamer | |

These low octanol/water partition coefficients demonstrate the hydrophilicity of the technetium-99m tricarbonyl-glycine and oligomers, indicating that after they are unbound, technetium-99m tricarbonyl-glycine and technetium-99m tricarbonyl-glycine oligomers do not remain within the body, and are excreted rapidly.

### Example 4

### SPECT/CT Imaging of Technetium-99m Tricarbonyl-Labeled Glycine Oligomer in Animal Model

To evaluate the in vivo behavior and distribution of the technetium-99m tricarbonyl glycine complexes, Micro-SPECT/CT images were taken. Labeling with the technetium-99m tricarbonyl glycine complexes was conducted in the same manner as in Example 1-2.

For Micro-SPECT/CT imaging, mice were scanned using the Inveon small-animal SPECT/CT system equipped with a single pinhole collimator (Siemens Medical Solutions, Knoxville, Tenn., USA). The mice were anesthetized with 2% isoflurane (prone position in a cradle) and intravenously injected with the technetium-99m tricarbonyl at a dose of 37 MBq 30 min before Micro-SPECT imaging. CT scanning was performed for 15 min using an X-ray source at 300 μA and 60 kV (one image per projection), with a 200 μm resolution. A total of 360 projections were obtained.

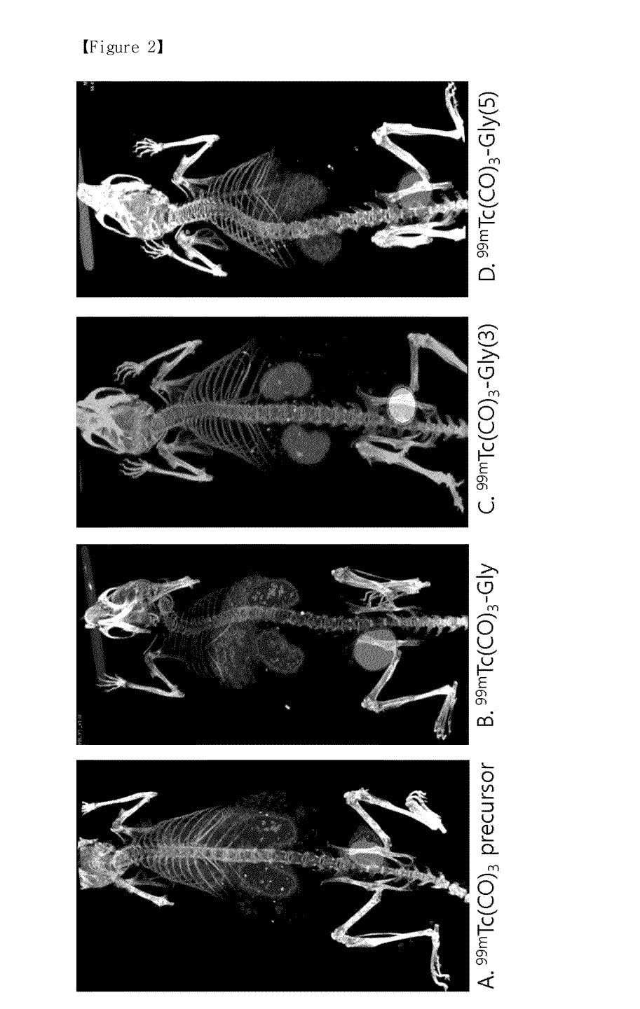

As shown in FIG. 2, the technetium-99m tricarbonyl-glycine trimer was observed to exhibit rapid clearance from blood, a high concentration in the kidney, and the highest accumulation rate with time in the bladder. Technetium-99m tricarbonyl-glycine and technetium-99m tricarbonyl-pentaglycine also showed rapid blood clearance, but some radioactivity was detected in the liver.

From the results, it is understood that all of the three technetium-99m tricarbonyl-glycine compounds are rapidly cleared through renal excretion, with the fastest blood clearance exhibited by technetium-99m tricarbonyl triglycine.

These data indicate that Gly-Gly-Gly (glycine trimer) is readily labeled with the technetium-99m tricarbonyl precursor and has excellent clearance.

Moreover, since Gly-Gly-Gly can be added to other peptide sequences, it finds various applications in the tomographic field. For example, ^{99m}Tc-mercaptoacety-Gly-Gly-Gly(MAG₃) may be used in monitoring renal functions by imaging.

Although the preferred embodiment(s) of the present invention have (has) been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The technetium-99m tricarbonyl-labeled glycine oligomers of the present invention can be useful as a radiotracer for gamma or SPECT imaging apparatus.

1. A method for preparing a technetium-99m-labeled monomer or oligomer, comprising:
a) synthesizing a technetium-99m tricarbonyl precursor; and
b) labeling a glycine monomer or oligomer with the technetium-99m tricarbonyl precursor.

2. The method of claim 1, wherein the glycine oligomer is a glycine trimer or a glycine pentamer.

3. The method of claim 1, wherein the glycine monomer or oligomer is further conjugated into a targeted peptide.

4. The method of claim 3, wherein the targeted peptide is selected from the group consisting of an RGD peptide (arginylglycylaspartic acid), neurotensin, somatostatin, angiotensin, luteinizing hormone releasing hormone, insulin, oxytocin, neurokinin-1, vasoactive intestinal peptide, substance P, neuropeptide Y, endothelin A, endothelin B, bradykinin, interleukin-1, epidermal growth factor, cholecystokinin, galanin, melanocyte-stimulating hormones, lanreotide, octreotide, and arginine-vasopressin.
